(19) United States
(12) Patent Application Publication
(10) Pub. No.: US 2024/0399010 A1
(43) Pub. Date: Dec. 5, 2024

(54) VOLATILE COMPOSITION DISPENSER WITH ACTIVATION LEVER

(71) Applicant: The Procter & Gamble Company, Cincinnati, OH (US)

(72) Inventors: William Michael CANNON, West Harrison, IN (US); Rahul VYAS, Singapore (SG); Bhavesh RAJWANI, Singapore (SG)

(21) Appl. No.: 18/678,489

(22) Filed: May 30, 2024

Related U.S. Application Data
(60) Provisional application No. 63/469,813, filed on May 30, 2023.

(51) Int. Cl. A61L 9/12; B65D 75/36

(52) U.S. Cl. CPC A61L 9/12; B65D 75/366; A61L 2209/133

(57) ABSTRACT

There is a volatile composition dispenser comprising a cartridge, a housing and an activation lever configured to be pivotable in relation to the housing. The activation lever is configurated to be pivotable between a first position and a second position to pierce a rupturable substrate of the cartridge to release at least one liquid volatile composition therefrom.

### TECHNICAL FIELD

The present disclosure relates to the field of devices and systems for delivering a volatile composition and particularly relates to a volatile composition dispenser with an activation lever, and a method of activating a volatile composition dispenser.

### BACKGROUND

Systems for delivering volatile materials to the atmosphere are well known in the art, and include for example, insect repellants, air fresheners, malodor removal agents. Such systems function by evaporating a volatile material through a medium such as a permeable membrane into a space to deliver a variety of benefits such as air freshening or malodor removal or a combination thereof. Typically, the volatile composition is stored in a sealed container that is opened or punctured to release the volatile composition to the air.

PCT Publication No. WO 98/16262 (hereinafter, “WO98/16262”) describes a disposable air freshener dispenser device having a push-button actuator which can be manually operated to initiate the dispensing of air freshener composition into the atmosphere. The device of WO98/16262 has an air freshener medium within a container, and a push button actuator which can be manually operated to rupture a foil covering the container for initiating the dispensing of the air freshener into the atmosphere.

However, after the foil is ruptured, the push button actuator stays on the openings within the foil and obstructs the path for dispensing the air freshener. Therefore, there exists a need that enables the foil to be ruptured without obstructing a dispensing of the air freshener.

A further problem associated with such devices is that it is difficult for a user (such as a consumer) to determine whether the air freshener device is activated until the consumer smells the air freshener composition. As a result, if the consumer does not smell the air freshener composition, the consumer may consider that the device is not activated or is malfunctioning, and this leads to reduced consumer satisfaction. Another problem of the prior art device is it is not easily detected by other users (such as retail store owners) whether such devices have been tampered with or inadvertently activated during handling or transportation to the retail stores. This may result in defective air freshener devices being displayed for sale which inevitably lead to consumer complaints when consumers purchase a defective air freshener device. Therefore, there exists a need for an apparatus for delivering a volatile material that can be manually operated and provides a signal to users indicating activation of the apparatus at the same time.

### SUMMARY

In order to address the above-identified needs, the present disclosure provides a volatile composition dispenser comprising:
(a) a cartridge comprising a reservoir of at least one liquid volatile composition and a rupturable substrate for enclosing the reservoir;
(b) a housing defining an interior compartment within which the cartridge is disposed, wherein a frame opening is formed through the housing and defines an extending surface in which the frame opening extends; and
(c) an activation lever disposed at the frame opening and configured to be pivotable in relation to the housing, wherein the activation lever comprises a hammer portion and a handle portion,

wherein the activation lever is configurated to be pivotable between a first position in which the hammer portion of the activation lever is extended below the extending surface of the frame opening and into the interior compartment of the housing and the handle portion of the activation lever is extended above the extending surface of the frame opening and outside the housing, and a second position in which the hammer portion of the activation lever causes rupture of the rupturable substrate of the cartridge to release the at least one liquid volatile composition therefrom.

### BRIEF DESCRIPTION OF THE DRAWINGS

While the specification concludes with the claims particularly pointing out and distinctly claiming the examples of the present disclosure, it is believed that the present disclosure will be better understood from the following description taken in conjunction with the accompanying drawings in which:

FIG. 1 is a front perspective view of a volatile composition dispenser according to an example;

FIG. 2 is a rear perspective view of the volatile composition dispenser shown in FIG. 1;

FIG. 3 is a side perspective exploded view of the volatile composition dispenser shown in FIG. 1;

FIG. 4 is a rear perspective view of a rupture mechanism of the volatile composition dispenser according to an example;

FIG. 5 is a rear perspective view of a frame opening and an activation lever of the volatile composition dispenser according to an example;

FIG. 6 is a bottom view of the frame opening and the activation lever of the volatile composition dispenser shown in FIG. 5;

FIGS. 7A-7C are sectional views of a volatile composition dispenser according to an example showing a first position, a second position and a third position of the activation lever respectively;

FIGS. 8A-8C are perspective view of the volatile composition dispenser according to an example showing a first position, a second position and a third position of the activation lever respectively;

FIGS. 9A-9C are sectional views of a volatile composition dispenser according to another example showing a first position, a second position and a third position of the activation lever respectively;

FIGS. 10A-10C are perspective view of the volatile composition dispenser according to another example showing a first position, a second position and a third position of the activation lever respectively; and

FIG. 11 is a sectional view of a volatile composition dispenser according to a further example showing different configuration of the activation lever.

### DETAILED DESCRIPTION

The present disclosure relates to a volatile composition dispenser for the delivery of a volatile material to the atmosphere. The dispenser is suitable for purposes of providing fragrances, air fresheners, deodorizers, odor eliminators, malodor counteractants, insecticides, insect repellants, medicinal substances, disinfectants, sanitizers, mood enhancers, aromatherapy aids, or for any other purpose using a volatile material or a volatile composition that acts to condition, modify, or otherwise change the atmosphere or the environment. For the purposes of illustrating the present disclosure in detail, but without intending to limit the scope of the present disclosure, the examples of the present disclosure will be described in a volatile composition dispenser for delivering a liquid composition containing perfume, perfume ingredients and/or perfume raw materials.

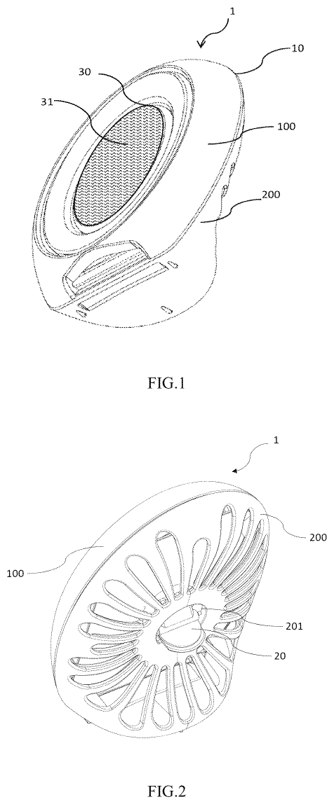

FIG. 1 shows a front perspective view of an example of a volatile composition dispenser 1 according to the present disclosure and FIG. 2 shows a rear perspective view of the dispenser 1. The volatile composition dispenser 1 comprises a housing 10 having a front cover 100 and a rear frame 200, the front cover 100 and the rear frame 200 defining an interior compartment. The rear frame 200 is provided with a frame opening 201 located substantially in the centre of the rear frame 200. An activation lever 20 is disposed within the frame opening 201 and is pivotable with respect to the rear frame 200 for enabling a user to activate the volatile composition dispenser 1. A cartridge 30 containing a volatile composition 31 is located within the housing 10.

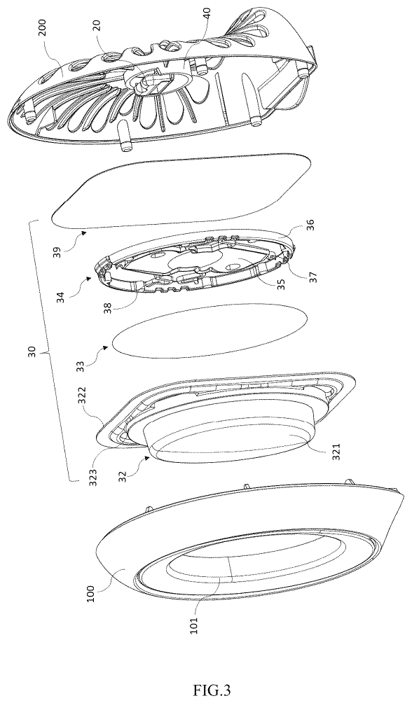

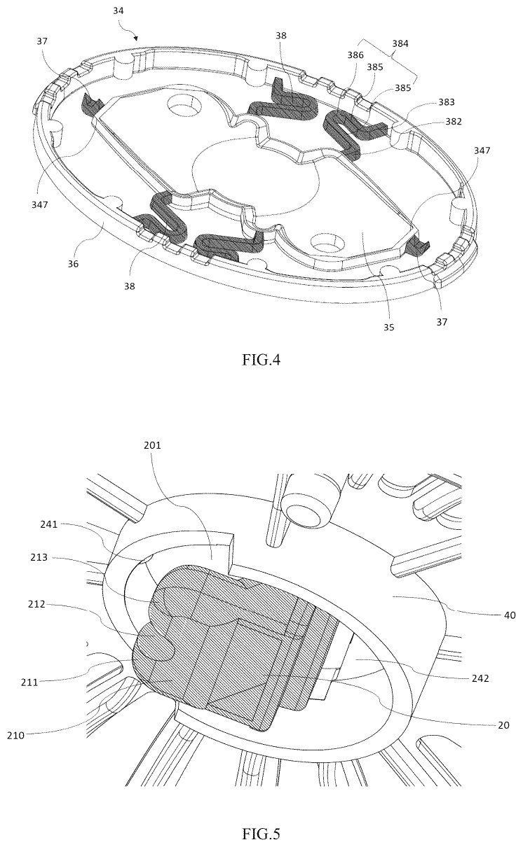

FIG. 3 shows internal components of the volatile composition dispenser 1. The front cover 100 comprises a window 101 configured for displaying the cartridge 30. The cartridge 30 comprises a container 32 having an orifice 321, within which the volatile composition 31 (as shown in FIG. 1) is stored. A rupturable substrate 33 is sealably attached to and covers the orifice 321 defining a reservoir to prevent the volatile composition 31 from being released until the volatile composition dispenser 1 is activated. The rupturable substrate 33 may be ruptured to release the volatile composition 31 by actuating a rupture mechanism 34 positioned adjacent to the rupturable substrate 33. The rupture mechanism 34 comprises a movable member 35 movably attached to an outer frame 36 by a resilient member 38. The resilient member 38 may be formed of one or more springs 38. One or more rupture elements 37 are arranged within the rupture mechanism 34 to puncture holes in the rupturable substrate 33. The rupture element 37 may be a pin, teeth or a tine. The cartridge 30 may comprise a breathable membrane 39 located on the exterior of the cartridge 30. The breathable membrane 39 may be scalably attached to a flange 322 located at a periphery 323 of the container 32. The breathable membrane 39 encloses the container 32, the volatile composition 31, the rupturable substrate 33, and the rupture mechanism 34. The breathable membrane 39 may be configured to flex when a pressure or an actuation force is applied on the breathable membrane 39.

FIG. 4 is a rear perspective view of the rupture mechanism 34 of the volatile composition dispenser 1. The rupture mechanism 34 comprises the outer frame 36 and the movable member 35 movably attached to the outer frame 36 by the resilient member 38. The movable member 35 comprises a substantially planar first surface facing the breathable membrane 39 and a second surface opposite the first surface and facing the rupturable substrate 33. The movable member 35 is movable relative to the outer frame 36 through flexing of the resilient member 38 when a force is received on the first surface of the movable member 35 during actuation of the activation lever 20. The resilient member 38 may be a spring resiliently connecting the outer frame 36 to the movable member 35.

In one example as shown in FIG. 4, a pair of rupture elements 37 are spaced apart on the movable member 35 and extend from opposing edges 347 of the movable member 35, wherein each rupture element 37 comprises a tip for puncturing the rupturable substrate 33. It could be understood that more than two rupture elements 37 or only one rupture element 37 could be provided and the rupture element 37 could be located at any suitable position on the movable member 35. For example, a first rupture elements 37 could be disposed on first opposing edges of the movable member 35 and a second pair of rupture elements 37 may be disposed on second opposing edges of the movable member 35 different from the first opposing edges to enable use of the cartridge 30 in different orientations. The position of the rupture elements 37 are configured to puncture the rupturable substrate 33 to allow air to enter the container 32 and drain the volatile composition from the container 32.

Further, in an example not shown, the movable member 35 may comprise one or more elongate ribs arranged to increase rigidity or stiffness of the movable member 35. For example, the elongate ribs may protrude from the second surface and extend from the edge of the movable member 35 and converge at the center of the movable member 35.

As shown in FIG. 4, the resilient member 38 may be a spring or a parallel spring system. The spring may be a beam or a torsional member. In an example, the resilient member 38 is a torsional may comprise a first arm 382 attached to the outer frame 36, a second arm 383 attached to the movable member 35, and an elongate channel member 384 extending between the first and second arms 382, 383. In an example, the elongate channel member 384 comprises two side beams 20 385 substantially parallel to each other and a bottom beam 386 so as to form a substantially U-shape.

To activate the volatile composition dispenser 1, a user depresses the activation lever 20 until it makes contact with and further push the rupture mechanism 34 (through the breathable membrane 39), and the rupture element 37 (such as a pin, teeth or a tine) on the rupture mechanism 34 pierce the rupturable substrate 33. Once the rupturable substrate 33 is pierced, the volatile composition 31 flows out of the container 32, wets the breathable membrane 39 and is then delivered to the atmosphere surroundings through evaporation from the breathable membrane 39.

According to an example of the present disclosure, the activation lever 20 and the rear frame 200 are configured to enable efficient and controlled rupturing of the rupturable substrate 33 in the cartridge 30 while additionally providing a tactile and intuitive user experience to the user for activating the volatile composition dispenser 1.

The frame opening 201 could be located substantially in the centre of the rear frame 200 and defines an extending surface 202 in which the frame opening 201 extends. The activation lever 20 is disposed within the frame opening 201 and is pivotable with respect to the rear frame 200. The frame opening 201 could be formed as a through hole in the rear frame 200. For example, according to an example of the present disclosure, an inner wall 40 could be provided at a periphery of the frame opening 201 and extend into the housing 10 from the interior of the rear frame 200. The inner wall 40 delimits the frame opening 201. In the example as shown, the inner wall 40 is solid and tubular in shape. However, the inner wall 40 may take some other shape such as for example a square cross section or a rectangular cross section. The inner wall 40 may be substantially cylindrical and comprise a continuous wall, or a segmented wall such as for example, a lattice structure or multiple elongate struts connected to one another. The inner wall 40 may define an extension of the frame opening 201 into the housing 10 with a central longitudinal axis running through the centre of the frame opening 201. The inner wall 40 could be configurated to allow the activation lever 20, at least a part of the activation lever 20, to pivot freely inside the inner wall 40. In the examples shown, the inner wall 40, the activation lever 20 and the rear frame 200 may comprise plastic for ease of manufacturing. Alternatively, the inner wall 40, the activation lever 20 and the rear frame 200 may comprise sheet metal, such as spring steel, and may be stamped or milled.

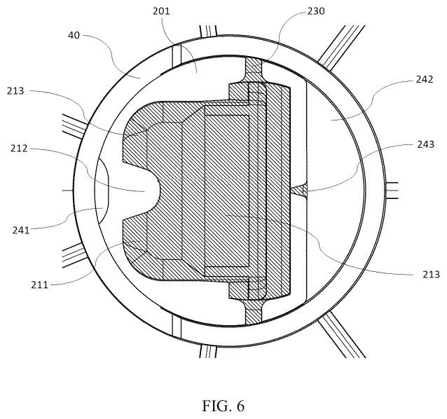

According to an example of the present disclosure, as shown in FIGS. 5-6, the activation lever 20 is connected to the rear frame 200 of the housing 10 at the frame opening 201 by a hinge structure 230 such that the activation lever 20 could be pivotable with respect to the rear frame 200. Further, the activation lever 20 could be connected pivotably to the inner wall 40 as shown in FIG. 6.

The hinge structure 230 is configurated to allow the activation lever 20 to be pivotable with respect to the rear frame 200. As shown in FIG. 6, the hinge structure 230 comprises a pivoting shaft located inside the frame opening 201 and coupled to the rear frame 200 of the housing 10, in particular to the inner wall 40. The pivoting shaft could be in form of a pin for example. According to one example, the pivoting shaft is secured to or integral with the rear frame 200 of the housing 10 and the activation lever 20 further comprises a through-hole into which the pivoting shaft fits, so that the activation lever 20 could be pivotable around the pivoting shaft and thus in relation to the rear frame 200 of the housing 10. According to another example, the rear frame 200 of the housing 10 further comprises at the frame opening 201 two blind-holes into which the pivoting shaft fits respectively, that is, each of two ends of the pivoting shaft fits into one of the two blind-holes, so that the pivoting shaft could be supported by the rear frame 200 of the housing 10 and rotatably fit within the two blind-holes. The activation lever 20 is secured to or integral with the pivoting shaft, so that the activation lever 20 could be pivotable in relation to the rear frame 200 of the housing 10 by means of the pivoting shaft.

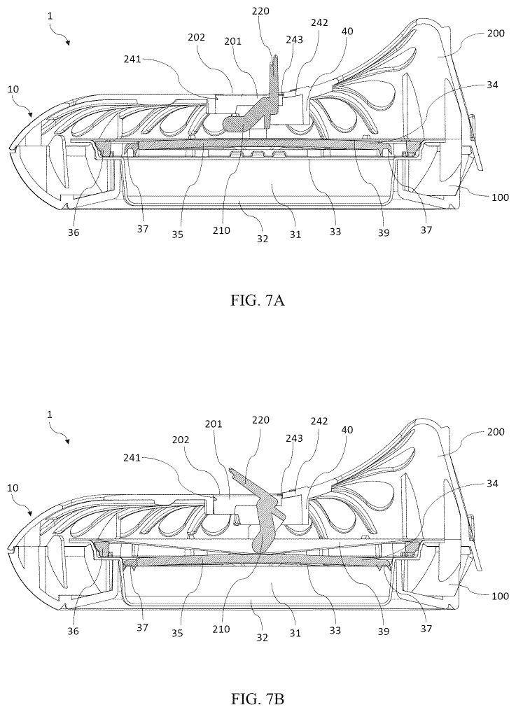

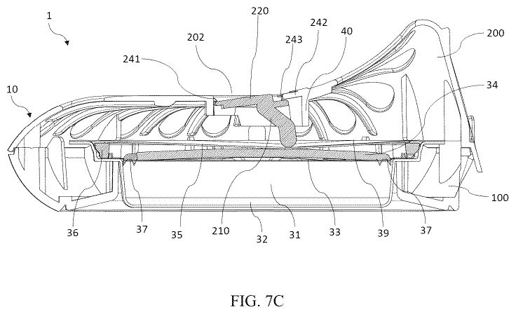

According to an example of the present disclosure, as shown in FIGS. 7A-11, the activation lever 20 comprises a hammer portion 210 for pushing the rupture mechanism 34 to pierce the rupturable substrate 33 and a handle portion 220 for being handled (such as being pushed) by a user. The hammer portion 210 and the handle portion 220 could be formed in one piece such that the activation lever 20 may be a unitary molded piece. Alternatively, the hammer portion 210 and the handle portion 220 could be formed separately and then coupled or secured to each other. the hammer portion 210 and the handle portion 220 could be coupled to each other at the hinge structure 230.

The handle portion 220 of the activation lever 20 could be in the form of such as a tab, a sheet, or a rod. Likewise, the hammer portion 210 of the activation lever 20 could be in the form of such as a tab, a sheet, or a rod. The hammer portion 210 of the activation lever 20 has an end opposite to the handle portion 220 in which the end could be rounded so as to facilitate the hammer portion 210 pushing the rupture mechanism 34 during the pivoting motion of the activation lever 20. In the example as shown in FIGS. 5 and 6, the end of the hammer portion 210 of the activation lever 20 comprises a first end 211 and a second end 213 which are separated apart from each other by a recess 212. The recess 212 is formed between the first end 211 and the second end 213.

According to an example of the present disclosure, the handle portion 220 of the activation lever 20 has a length of from 20% to 150%, or from 50% to 120%, or from 70% to 100% of a length of the hammer portion 210 of the activation lever 20. It is desirable that the handle portion is not disproportionally longer or larger than the hammer portion, so that the protruding handle portion, while providing a clear visual signal to the consumer of the pre-activation status of the device, does not result in significant spatial hinderance during shipment or storage. The length of the handle portion 220 is a length in the direction of the handle portion 220 extending from the connection between the hammer portion 210 and the handle portion 220, such as the length of the handle portion 220 extending in the page of FIG. 7A-7C. Likewise, the length of the hammer portion 210 is a length in the direction of the hammer portion 210 extending from the connection between the hammer portion 210 and the handle portion 220, such as the length of the hammer portion 210 extending in the page of FIG. 7A-7C.

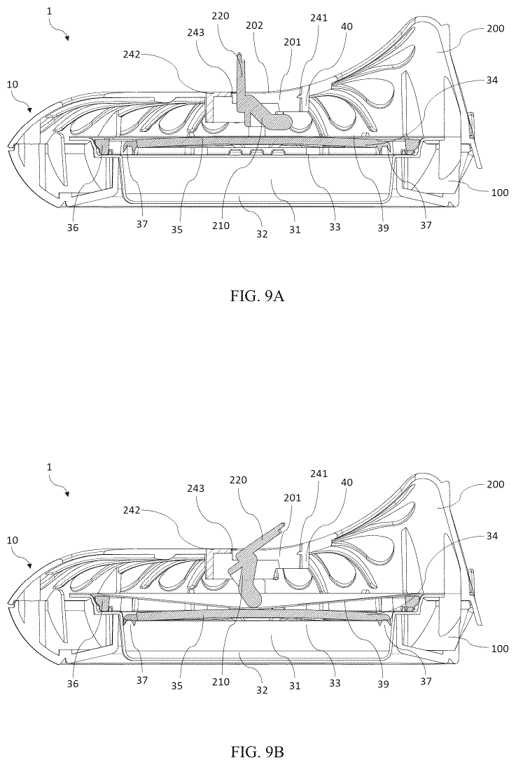

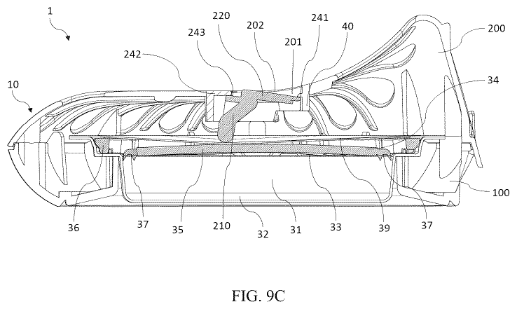

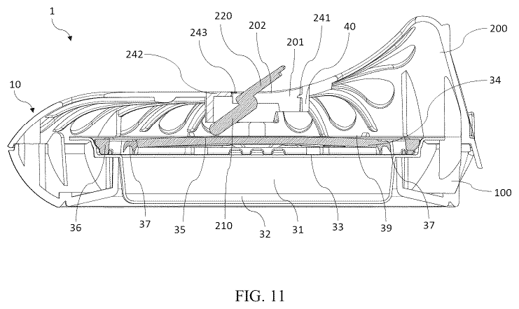

According to an example of the present disclosure, the handle portion 220 and the hammer portion 210 of the activation lever 20 form an angle of from 90° to 180°, or from 100° to 170°, or from 120° to 160°, or from 130° to 150°, or preferably 145°. In general, such angle could be considered as an angle formed by the length extension of the hammer portion 210 and the length extension of the handle portion 220. As shown in FIGS. 7A-7C and 9A-9C, the angle formed by the handle portion 220 and the hammer portion 210 is an obtuse angle, and as shown in FIG. 11, the angle formed by the handle portion 220 and the hammer portion 210 is a straight angle in which the length extension of the hammer portion 210 is in line with or aligned with the length extension of the handle portion 220. In a specific example, the angle between the handle portion 220 and the hammer portion 210 is an obtuse angle (similar to that shown in FIG. 11), which allows the handle portion 220 to start from an upright pre-activation position and ends at a flushed or substantial parallel post-activation position in relation to the rear frame 200, while the hammer portion can effectively move from a slated pre-activation position into a downright activation position and ends at a slanted post-activation position in relation to the rear frame 200 (as shown in FIGS. 7A-7C.

The above description about the size, shape, and structure of the activation lever 20 is only for illustrative and not limiting. It will be understood by those skilled in the art that the activation lever 20 could take any suitable size, shape, and structure without departing from the spirit and scope of the present disclosure.

According to an example of the present disclosure, the activation lever 20 is configurated to be pivotable between a first position and a second position. In the first position, as shown in FIGS. 7A, 8A, 9A and 10A, the hammer portion 210 of the activation lever 20 is extended below the extending surface 202 of the frame opening 201 and into the interior compartment of the housing 10, and the handle portion 220 of the activation lever 20 is extended above the extending surface 202 of the frame opening 201 and outside the housing 10. The handle portion 220 of the activation lever 20 protrudes beyond the housing providing a clear visual cue to the user that the volatile composition dispenser 1 is currently in a pre-activation state, i.e., the volatile composition dispenser 1 is not activated. In addition, this configuration of the handle portion 220 facilitates the user to push on the handle portion 220 to activate the volatile composition dispenser 1. In the second position, as shown in FIGS. 7B, 8B, 9B and 10B, the hammer portion 210 of the activation lever 20 causes rupture of the rupturable substrate 33 of the cartridge 30 to release the at least one liquid volatile composition 31 therefrom. The terms of “above” and “below” herein are defined based on the orientation of the components in the figures, such as FIGS. 7A-7C and 9A-9C. When the activation lever 20 is pushed by the user to pivot from the first position to the second position, the handle portion 220 of the activation lever 20 is pushed and thus the activation lever 20 is pivoted around the pivoting shaft of the hinge structure 230 and thus in relation to the rear frame 200 of the housing 10. The hammer portion 210 and the handle portion 220 of the activation lever 20 are both pivoted around the pivoting shaft. During the activation lever 20 is pivoted from the first position to the second position, the hammer portion 210 of the activation lever 20 will exert a force on the rupture mechanism 34 and moves the rupture mechanism 34 to rupture the rupturable substrate 33 in the cartridge 30 and to release the at least one liquid volatile composition 31 therefrom.

According to an example of the present disclosure, as shown in FIGS. 7A-10C, during the motion of the activation lever 20, the position of the activation lever 20 with respect to the rear frame 200 of the housing 10 will be changed, and the angle of the hammer portion 210 and the handle portion 220 of the activation lever 20 with respect to the extending surface 202 in which the frame opening 201 extends will also be changed. In the first position, as shown in FIGS. 7A and 9A, the handle portion 220 of the activation lever 20 extends away from the extending surface 202 at an angle of from 30° to 150°, or from 45° to 135°, or from 60° to 120°, or preferably 90°, and the hammer portion 210 of the activation lever 20 extends away from the extending surface 202 at an angle of from 10° to 80°, or from 20° to 60°, or from 30° to 40°, or preferably 35°. In the second position, as shown in FIGS. 7B and 9B, the hammer portion 210 of the activation lever 20 will be changed to extend away from the extending surface 202 at an angle of from 70° to 110°, or from 80° to 100°, or from 85° to 95°, or preferably 90°.

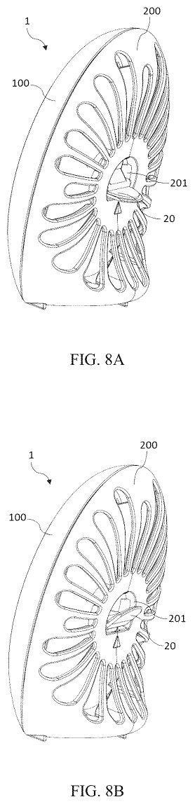

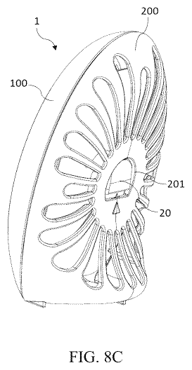

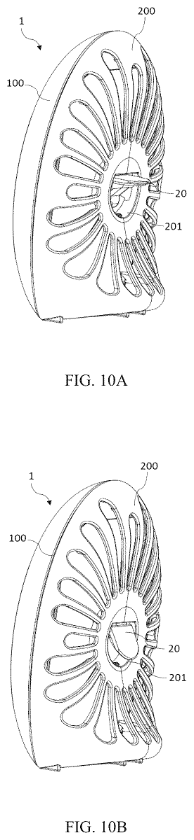

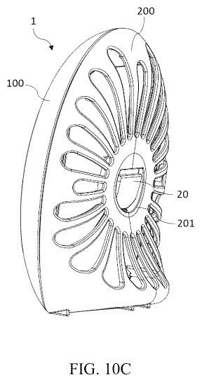

According to an example of the present disclosure, as shown in FIGS. 7A-10C, the activation lever 20 is further configurated to be pivotable from the second position to a third position in which the handle portion 220 of the activation lever 20 extends in a direction that is substantially parallel to the extending surface 202 of the frame opening 201, as shown in FIGS. 7C, 8C, 9C and 10C. In this case, the activation lever 20 could be further pivotable after reaching the second position and beyond the second position. In the third position, the handle portion 220 of the activation lever 20 is substantially parallel to the extending surface 202 of the frame opening 201, which provides a clear visual cue to the user that the volatile composition dispenser 1 is currently in a post-activation state, i.e., the volatile composition dispenser 1 has been completely activated. In addition, this configuration of the handle portion 220 provides an aesthetically pleasing appearance of the volatile composition dispenser 1, as shown in FIGS. 8C and 10C. The term of “substantially parallel to” indicates that the angle formed between the handle portion 220 of the activation lever 20 and the extending surface 202 of the frame opening 201 may be in a range of −15° to 15°. In the third position, the hammer portion 210 of the activation lever 20 extends away from the extending surface 202 at an angle of from 45° to 70°, or from 50° to 65°, or from 55° to 60°, or preferably 60°.

According to an example of the present disclosure, the activation lever 20 could be locked in the third position once it reaches this position. As shown in FIGS. 5-6, the volatile composition dispenser 1 comprises a first stop 241 and a second stop 242. The first stop 241 and the second stop 242 may be both located within the frame opening 201 and preferably opposite to each other. As shown in FIG. 6, the first stop 241 could be opposite to the second stop 242 in a direction perpendicular to the pivoting shaft of the hinge structure 230. In the third position, the first stop 241 could be interacted with an end of the handle portion 220 of the activation lever 20 to prevent the activation lever 20 pivoting backwardly from the third position such as to the second position or the first position, and the second stop 242 could be interacted with the hammer portion 210 of the activation lever 20 to prevent the activation lever 20 further pivoting from the third position, such as beyond the third position, so that the activation lever 20 is locked in the third position.

The first stop 241 could be provided on the inner wall 40 and in the form of a protrusion which protrudes from the inner wall 40 towards the centre of the frame opening 201. In one example, the first stop 241 could form a snap-fit with the end of the handle portion 220 of the activation lever 20. During the activation lever 20 pivoting between the first position and the second position, the first stop 241 will be not contacted with the end of the handle portion 220 of the activation lever 20. During the activation lever 20 pivoting from the second position to the third position, the end of the handle portion 220 will be contacted with the first stop 241 and it is needed for the handle portion 220 of the activation lever 20 to overcome the interaction between the end of the handle portion 220 and the first stop 241 to move beyond the first stop 241. After the handle portion 220 of the activation lever 20 moving beyond the first stop 241 and the activation lever 20 reaching its third position, the first stop 241 is configurated to prevent the activation lever 20 pivoting backwardly from the third position such as to the second position and/or the first position, as shown in FIGS. 7C and 9C.

The second stop 242 could be provided on the inner wall 40 and in the form of a platform or a block which extends from the inner wall 40 towards the centre of the frame opening 201. During the activation lever 20 pivoting between the first position and the second position, the second stop 242 will be not contacted with the hammer portion 210 of the activation lever 20. As the activation lever 20 pivoting from the second position to the third position, the hammer portion 220 will be contacted with the second stop 242 and blocked by the second stop 242, so as to prevent the activation lever 20 further pivoting from the third position, such as beyond the third position, as shown in FIGS. 7C and 9C. Therefore, the activation lever 20 could be locked in the third position through the combination of the first stop 241 and the second stop 242.

Generally, it is desired for the activation lever 20 to be kept in its first position before used by a user. In fact, it may be preferable that the activation lever 20 could be kept in the first position to prevent it from being inadvertently actuated and thus activating the volatile composition dispenser 1, but be able to be easily pushed to activate the volatile composition dispenser 1 while the user want to use it. According to an example of the present disclosure, a fixation element 243 is provided to achieve such effect. The activation lever 20 is stabilized to the rear frame 200 of the housing 10 and held in the first position by the fixation element 243. The fixation element 243 is connected between the activation lever 20 and the inner wall 40. Alternatively, in the case of the second stop 242, the fixation element 243 could be connected between the activation lever 20 and the second stop 242, as shown in FIGS. 5 and 6. Upon the activation lever 20 pivoting from the first position to the second position, the fixation element 243 is removed or broken to enable movement of the activation lever 20. The fixation element 243 could be broken by the application of a pressure onto the handle portion 220 of the activation lever 20. Such pressure could be created by the user pushing the handle portion 220 of the activation lever 20, and the activation lever 20 could pivot from the first position to the second position through such pressure. For example, the fixation element 243 could be in the form of a tab or a sheet provided with a weakness line which would be broken upon the user pushing the handle portion 220 of the activation lever 20.

The operation of the volatile composition dispenser 1 according to the present disclosure will be described below with reference to FIGS. 7A-10C.

The volatile composition dispenser 1 comprises a pre-activation state, an activation state, and a post-activation state. FIGS. 7A and 8A show the pre-activation state of the volatile composition dispenser 1. In the pre-activation state, the activation lever 20 is stabilized to the rear frame 200 of the housing 10 and held in the first position by the fixation element 243. The hammer portion 210 of the activation lever 20 is extended below the extending surface 202 of the frame opening 201 and into the interior compartment of the housing 10, and the handle portion 220 of the activation lever 20 is extended above the extending surface 202 of the frame opening 201 and outside the housing 10, which provides a clear visual cue to the user that the volatile composition dispenser 1 is currently in the pre-activation state. The hammer portion 210 of the activation lever 20 could be contacted with the breathable membrane 39 or separated from the breathable membrane 39 by a distance. The rupture mechanism 34 is sandwiched between the breathable membrane 39 and the rupturable substrate 33, and not pushed by the hammer portion 210 of the activation lever 20 to contact and pierce the rupturable substrate 33.

When a user wants to activate the volatile composition dispenser 1 to release the at least one liquid volatile composition 31 therefrom, he/she could push the handle portion 220 of the activation lever 20, for example, push the handle portion 220 towards the left side in FIGS. 7A-7C, so that the activation lever 20 is pivoted from the first position to the second position so as to activate the volatile composition dispenser 1 from the pre-activation state to the activation state. FIGS. 7B and 8B show the activation state of the volatile composition dispenser 1. During the process from the pre-activation state to the activation state, the fixation element 243 is broken through pushing the handle portion 220 of the activation lever 20, and then the activation lever 20 is pivoted from the first position to the second position. In the activation state, the hammer portion 210 of the activation lever 20 is pivoted to apply a pressure onto the breathable membrane 39 which will be flexed towards the rupture mechanism 34, and then apply a pressure onto the rupture mechanism 34, in particular the movable member 35 thereof. The rupture mechanism 34, in particular the movable member 35 thereof, will move towards and pierce the rupturable substrate 33 with the rupture element 37 under such pressure. In some case, the rupturable substrate 33 may also be flexed downwardly following the downward movement of the movable member 35 of the rupture mechanism 34. The at least one liquid volatile composition 31 could be released from the container 32 through the holes formed in the rupturable substrate 33 by the rupture element 37 of the rupture mechanism 34 piercing the rupturable substrate 33.

With continue pushing the handle portion 220 of the activation lever 20, the activation lever 20 will be further pivoted from the second position to the third position so as to put the volatile composition dispenser 1 from the activation state to the post-activation state, during which the pressure applied by the hammer portion 210 of the activation lever 20 onto the rupture mechanism will be reduced with the further pivoting of the activation lever 20. The rupture mechanism 34 could move backwardly (upwardly) due to the clastic force of the resilient member 38. In this case, the rupturable substrate 33 and the breathable membrane 39 could be flexed adaptively.

By means of the activation lever 20 pivoting from the first position, through the second position and to the third position, a click effect is obtained in the case of the user pushing the handle portion 220 of the activation lever 20, which provides the user with a wonderful sensation.

FIGS. 7C and 8C show the post-activation state of the volatile composition dispenser 1. In the post-activation state, the handle portion 220 of the activation lever 20 extends in a direction that is substantially parallel to the extending surface 202 of the frame opening 201 providing an aesthetically pleasing appearance of the volatile composition dispenser 1. As shown in FIG. 7C, the hammer portion 210 of the activation lever 20 is still contacted with the breathable membrane 39 and applies a pressure onto the breathable membrane 39 and the rupture mechanism 34. The rupture mechanism 34, in particular the movable member 35 thereof, is only partially returned but not completely returned to its original position. In this case, the holes formed in the rupturable substrate 33 are large enough and could be partially exposed. Alternatively, in an example not shown, in the post-activation state, the hammer portion 210 of the activation lever 20 could be separated from the breathable membrane 39 and no longer apply a pressure onto the rupture mechanism 34. In this case, the holes formed in the rupturable substrate 33 could be totally exposed to release the at least one liquid volatile composition 31. Due to the at least partially return of the movable member 35 of the rupture mechanism 34, the holes formed in the rupturable substrate 33 could be at least partially exposed to facilitate the release of the at least one liquid volatile composition 31.

FIGS. 9A-10C show a similar example as in FIGS. 7A-8C, except for that the activation lever 20 is pivoted in an opposite direction than that in FIGS. 7A-8C. The handle portion 220 is pushed towards the left side in FIGS. 7A-7C, and the handle portion 220 is pushed towards the right side in FIGS. 9A-9C.

The volatile composition dispenser 1 may comprise a small form factor such as a form factor similar to a computer mouse so as for ergonomic fit in the hand of the user and ease of use. The internal components of the cartridge 30 as shown in FIG. 3 may be characterized as follows. For example, dimensions of the container 32 may be configured to hold about 1 ml to about 50 ml of a liquid volatile composition. Alternatively, the reservoir may hold about 2 ml to about 30 ml, alternatively about 2 ml to about 10 ml, alternatively about 2 ml to about 8 ml, alternatively about 4 ml to about 6 ml, alternatively about 2 ml, alternatively about 6 ml of a liquid volatile composition. Further, a shape of the container 32 may be configured to correspond to a shape of the opening 101 of the front cover 100. For example, the container 32 may define a substantially elliptical or oval shape and its width to length ratio may be about 1:2 to 1:2.5.

The rupturable substrate 33 could be made of any material that ruptures with applied force, with or without the presence of an element to aid in such rupture. Because the rupturable substrate 33 is intended to contain a volatile material while in storage, it may be made from any barrier material that prevents evaporation of the volatile material prior to its intended use. Such materials may be impermeable to vapors and liquids. Suitable barrier materials for the rupturable substrate 33 include a flexible film, such as a polymeric film, a flexible foil, or a composite material such as foil/polymeric film laminate. Suitable flexible foils include a metal foil such as a foil comprised of a nitrocellulose protective lacquer, a 20 micron aluminum foil, a polyurethane primer, and 15 g/m² polyethylene coating (Lidfoil 118-0092), available from Alcan Packaging. Suitable polymeric films include polyethylene terephtalate (PET) films, acrylonitrile copolymer barrier films such as those sold under the tradename Barex® by INOES, ethylene vinyl alcohol, and combinations thereof. It is also contemplated that coated barrier films may be utilized as a rupturable substrate 33. Such coated barrier films include metallized PET, metalized polypropylene, silica or alumina coated film may be used. Any barrier material, whether coated or uncoated, may be used alone and or in combination with other barrier materials.

The rupture element 37 could be injection, compression, or pressure molded using a polyolefin, such as polyethylene or polypropylene; polyester; or other plastics known to be suitable for molding. The rupture element 37 could also be made by thermoforming with a discrete cutting step to remove parts not wanted.

The breathable membrane 39 may have an average pore size of about 0.01 to about 0.06 microns, alternatively from about 0.01 to about 0.05 microns, alternatively about 0.01 to about 0.04 microns, alternatively about 0.01 to about 0.03 microns, alternatively about 0.02 to about 0.04 microns, alternatively about 0.02 microns. Further, the breathable membrane 39 may be filled with any suitable filler and plasticizer known in the art. Fillers may include finely divided silica, clays, zeolites, carbonates, charcoals, and mixtures thereof. The microporous breathable membrane 39 may be filled with about 50% to about 80%, by total weight, of silica, alternatively about 60% to about 80%, alternatively about 70% to about 80%, alternatively about 70% to about 75%. A thickness of the breathable membrane 39 may be about 0.01 mm to about 1 mm, alternatively between about 0.1 mm to 0.4 mm, alternatively about 0.15 mm to about 0.35 mm, alternatively about 0.25 mm.

Still further, an evaporative surface area of the microporous breathable membrane 39 may be about 2 cm² to about 100 cm², alternatively about 2 cm² to about 25 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm². Accordingly, the rear frame 200 may be sized and shaped to fit the evaporative surface area of the breathable membrane 39.

Suitable microporous membranes for the present disclosure include a microporous, ultra-high molecular weight polyethylene (UHMWPE) optionally filled with silica as described in U.S. Pat. No. 7,498,369. Such UHMWPE microporous membranes include Daramic™ V5, available from Daramic, Solupor®, available from DSM (Netherlands), and Teslin™, available from PPG Industries, and combinations thereof.

A volatile material or composition suitable for use in the cartridge 30 for a volatile composition dispenser 1 may be configured to condition, modify, or otherwise change the atmosphere and may include compositions suitable for the purposes of providing fragrances, air fresheners, deodorizers, odor eliminators, malodor counteractants, insecticides, insect repellants, medicinal substances, disinfectants, sanitizers, mood enhancers, and aromatherapy aids. A list of the suitable volatile materials is shown in Table 1 below.

TABLE 1

| Purpose | Volatile Material |
|---|---|
| Providing | Perfume oil, volatile essential oils, volatile organic |
| fragrances | compound, synthetically or naturally formed materials. |
| | Examples include, but are not limited to: oil of bergamot, |
| | bitter orange, lemon, mandarin, caraway, cedar leaf, clove |
| | leaf, cedar wood, geranium, lavender, orange, origanum, |
| | petitgrain, white cedar, patchouli, neroili, rose absolute, and |
| | the like. |
| | Suitable crystalline solids include but are not limited to: |
| | vanillin, ethyl vanillin, coumarin, tonalid, calone, |
| | heliotropene, musk xylol, cedrol, musk ketone benzohenone, |
| | raspberry ketone, methyl naphthyl ketone beta, phenyl ethyl |
| | salicylate, veltol, maltol, maple lactone, proeugenol acetate, |
| | evemyl, and the like. |
| Neutralize | Suitable malodor compositions include reactive aldehydes |
| malodors | and ionones |

The composition may be formulated such that the composition comprises a volatile material mixture comprising about 10% to about 100%, by total weight, of volatile materials that each having a VP at 25° C. of less than about 0.01 torr; alternatively about 40% to about 100%, by total weight, of volatile materials each having a VP at 25° C. of less than about 0.1 torr; alternatively about 50% to about 100%, by total weight, of volatile materials each having a VP at 25° C. of less than about 0.1 torr; alternatively about 90% to about 100%, by total weight, of volatile materials each having a VP at 25° C. of less than about 0.3 torr. The volatile material mixture may include 0% to about 15%, by total weight, of volatile materials each having a VP at 25° C. of about 0.004 torr to about 0.035 torr; and 0% to about 25%, by total weight, of volatile materials each having a VP at 25° C. of about 0.1 torr to about 0.325 torr; and about 65% to about 100%, by total weight, of volatile materials each having a VP at 25° C. of about 0.035 torr to about 0.1 torr. One source for obtaining the saturation vapor pressure of a volatile material is EPI Suite™, version 4.0, available from U.S. Environmental Protection Agency.

An example is shown below:
A. A volatile composition dispenser (1), comprising:
   (a) a cartridge (30) comprising a reservoir of at least one liquid volatile composition (31) and a rupturable substrate (33) for enclosing the reservoir;
   (b) a housing (10) defining an interior compartment within which the cartridge (30) is disposed, wherein a frame opening (201) is formed through the housing (10) and defines an extending surface (202) in which the frame opening (201) extends; and
   (c) an activation lever (20) disposed at the frame opening (201) and configured to be pivotable in relation to the housing (10), wherein the activation lever (20) comprises a hammer portion (210) and a handle portion (220),
   wherein the activation lever (20) is configurated to be pivotable between a first position in which the hammer portion (210) of the activation lever (20) is extended below the extending surface (202) of the frame opening (201) and into the interior compartment of the housing (10) and the handle portion (220) of the activation lever (20) is extended above the extending surface (202) of the frame opening (201) and outside the housing (10), and a second position in which the hammer portion (210) of the activation lever (20) causes rupture of the rupturable substrate (33) of the cartridge (30) to release the at least one liquid volatile composition (31) therefrom.
B. The volatile composition dispenser (1) according to paragraph A, wherein the activation lever (20) is configurated to be pivotable from the second position to a third position in which the handle portion (220) of the activation lever (20) extends in a direction that is substantially parallel to the extending surface (202) of the frame opening (201).
C. The volatile composition dispenser (1) according to paragraph B, wherein the activation lever (20) is configurated to be lockable in the third position.
D. The volatile composition dispenser (1) according to paragraph C, further comprising a first stop (241) and a second stop (242) both located within the frame opening (201) and opposite to each other, wherein in the third position, the first stop (241) could be interacted with an end of the handle portion (220) of the activation lever (20) to prevent the activation lever (20) pivoting backwardly from the third position and the second stop (242) could be interacted with the hammer portion (210) of the activation lever (20) to prevent the activation lever (20) further pivoting from the third position, so that the activation lever (20) is locked in the third position.
E. The volatile composition dispenser (1) according to paragraph A, wherein the activation lever (20) is connected to the housing (10) at the frame opening (201) by a hinge structure (230).
F. The volatile composition dispenser (1) according to paragraph E, wherein the hinge structure (230) comprises a pivoting shaft located inside the frame opening (201) and coupled to the housing (10).
G. The volatile composition dispenser (1) according to paragraph F, wherein the activation lever (20) further comprises a through-hole into which the pivoting shaft fits, so that the activation lever (20) could be pivotable around the pivoting shaft and in relation to the housing (10).
H. The volatile composition dispenser (1) according to paragraph F, wherein the housing (10) further comprises at the frame opening (201) two blind-holes into which the pivoting shaft fits respectively, and the activation lever (20) is secured to or integral with the pivoting shaft, so that the activation lever (20) could be pivotable in relation to the housing (10) by means of the pivoting shaft.
I. The volatile composition dispenser (1) according to paragraph A, wherein the activation lever (20) may be a unitary molded piece.
J. The volatile composition dispenser (1) according to paragraph A, wherein the handle portion (220) of the activation lever (20) has a length of from 20% to 150%, or from 50% to 120%, or from 70% to 100% of that of the hammer portion (210) of the activation lever (20).
K. The volatile composition dispenser (1) according to paragraph A, wherein the handle portion (220) and the hammer portion (210) of the activation lever (20) form an angle of from 90° to 180°, or from 100° to 170°, or from 120° to 160°, or from 130° to 150°, or preferably 145°.
L. The volatile composition dispenser (1) according to paragraph A, wherein in the first position, the handle portion (220) of the activation lever (20) extends away from the extending surface (202) at an angle of from 30° to 150°, or from 45° to 135°, or from 60° to 120°, or preferably 90°.
M. The volatile composition dispenser (1) according to paragraph A, wherein in the first position, the hammer portion (210) of the activation lever (20) extends away from the extending surface (202) at an angle of from 10° to 80°, or from 20° to 60°, or from 30° to 40°, or preferably 35°.
N. The volatile composition dispenser (1) according to paragraph A, wherein in the second position, the hammer portion (210) of the activation lever (20) extends away from the extending surface (202) at an angle of from 70° to 110°, or from 80° to 100°, or from 85° to 95°, or preferably 90°.
O. The volatile composition dispenser (1) according to paragraph B, wherein in the third position, the hammer portion (210) of the activation lever (20) extends away from the extending surface (202) at an angle of from 45° to 70°, or from 50° to 65°, or from 55° to 60°, or preferably 60°.
P. The volatile composition dispenser (1) according to paragraph A, wherein the activation lever (20) is stabilized to the housing (10) and held in the first position by a fixation element (243), and upon the activation lever (20) pivoting from the first position to the second position, the fixation element (243) is removed or broken to enable movement of the activation lever (20).
Q. The volatile composition dispenser (1) according to paragraph P, wherein the fixation element (243) is broken by the application of a pressure onto the handle portion (220) of the activation lever (20) through which pressure the activation lever (20) could pivot from the first position to the second position.
R. The volatile composition dispenser (1) according to paragraph A, wherein the cartridge (30) further comprises a breathable membrane (39) for enclosing the reservoir and evaporating the at least one liquid volatile composition (31) into the atmosphere.
S. The volatile composition dispenser (1) according to paragraph R, further comprising a rupture mechanism (34) sandwiched between the rupture substrate (33) and the breathable membrane (39), wherein upon the activation lever (20) pivoting from the first position to the second position, the hammer portion (210) of the activation lever (20) exerts a force on the rupture mechanism (34) and moves the rupture mechanism (34) to rupture the rupturable substrate (33) in the cartridge (30) and to release the at least one liquid volatile composition (31) therefrom.
T. A method of delivering at least one liquid volatile composition into the atmosphere, the method comprising:
   (i) providing a volatile composition dispenser (1) according to any one of the preceding claims; and
   (ii) activating the volatile composition dispenser (1) by applying pressure onto the handle portion (220) of the activation lever (20).
U. The method according to paragraph T, wherein activating the volatile composition dispenser (1) comprises pivoting the activation lever (20) from the first position to the second position by applying pressure onto the handle portion (220) of the activation lever (20).

Various examples will now be described to provide an overall understanding of the principles of the structure, function, manufacture, and use of the apparatuses and methods disclosed herein. One or more examples of these examples are illustrated in the accompanying drawings. Those of ordinary skill in the art will understand that the apparatuses and methods specifically described herein and illustrated in the accompanying drawings are non-limiting example examples and that the scope of the various examples of the present disclosure are defined solely by the claims. The features illustrated or described in connection with one example may be combined with the features of other example examples. Such modifications and variations are intended to be included within the scope of the present disclosure.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any examples disclosed or claimed herein or that it alone, or in any combination with any other reference or references, teaches, suggests or discloses any such example. Further, to the extent that any meaning or definition of a term in this document conflicts with any meaning or definition of the same term in a document incorporated by reference, the meaning or definition assigned to that term in this document shall govern.

While particular examples of the present disclosure have been illustrated and described, it would be obvious to those skilled in the art that various other changes and modifications could be made without departing from the spirit and scope of the present disclosure. It is therefore intended to cover in the appended claims all such changes and modifications that are within the scope of the present disclosure.

What is claimed is:

1. A volatile composition dispenser, comprising:
(a) a cartridge comprising a reservoir of at least one liquid volatile composition and a rupturable substrate for enclosing the reservoir;
(b) a housing defining an interior compartment within which the cartridge is disposed, wherein a frame opening is formed through the housing and defines an extending surface in which the frame opening extends; and
(c) an activation lever disposed at the frame opening and configured to be pivotable in relation to the housing, wherein the activation lever comprises a hammer portion and a handle portion,
wherein the activation lever is configurated to be pivotable between a first position in which the hammer portion of the activation lever is extended below the extending surface of the frame opening and into the interior compartment of the housing and the handle portion of the activation lever is extended above the extending surface of the frame opening and outside the housing, and a second position in which the hammer portion of the activation lever causes rupture of the rupturable substrate of the cartridge to release the at least one liquid volatile composition therefrom.

2. The volatile composition dispenser according to claim 1, wherein the activation lever is configurated to be pivotable from the second position to a third position in which the handle portion of the activation lever extends in a direction that is substantially parallel to the extending surface of the frame opening.

3. The volatile composition dispenser according to claim 2, wherein the activation lever is configurated to be lockable in the third position.

4. The volatile composition dispenser according to claim 3, further comprising a first stop and a second stop both located within the frame opening and opposite to each other, wherein in the third position, the first stop could be interacted with an end of the handle portion of the activation lever to prevent the activation lever pivoting backwardly from the third position and the second stop could be interacted with the hammer portion of the activation lever to prevent the activation lever further pivoting from the third position, so that the activation lever is locked in the third position.

5. The volatile composition dispenser according to claim 1, wherein the activation lever is connected to the housing at the frame opening by a hinge structure.

6. The volatile composition dispenser according to claim 5, wherein the hinge structure comprises a pivoting shaft located inside the frame opening and coupled to the housing.

7. The volatile composition dispenser according to claim 6, wherein the activation lever further comprises a through-hole into which the pivoting shaft fits, so that the activation lever could be pivotable around the pivoting shaft and in relation to the housing.

8. The volatile composition dispenser according to claim 6, wherein the housing further comprises at the frame opening two blind-holes into which the pivoting shaft fits respectively, and the activation lever is secured to or integral with the pivoting shaft, so that the activation lever could be pivotable in relation to the housing by means of the pivoting shaft.

9. The volatile composition dispenser according to claim 1, wherein the activation lever may be a unitary molded piece.

10. The volatile composition dispenser according to claim 1, wherein the handle portion of the activation lever has a length of from 20% to 150% of that of the hammer portion of the activation lever.

11. The volatile composition dispenser according to claim 1, wherein the handle portion and the hammer portion of the activation lever form an angle of from 90° to 180°.

12. The volatile composition dispenser according to claim 1, wherein in the first position, the handle portion of the activation lever extends away from the extending surface at an angle of from 30° to 150°.

13. The volatile composition dispenser according to claim 1, wherein in the first position, the hammer portion of the activation lever extends away from the extending surface at an angle of from 10° to 80°.

14. The volatile composition dispenser according to claim 1, wherein in the second position, the hammer portion of the activation lever extends away from the extending surface at an angle of from 70° to 110°.

15. The volatile composition dispenser according to claim 2, wherein in the third position, the hammer portion of the activation lever extends away from the extending surface at an angle of from 45° to 70°.

16. The volatile composition dispenser according to claim 1, wherein the activation lever is stabilized to the housing and held in the first position by a fixation element, and upon the activation lever pivoting from the first position to the second position, the fixation element is removed or broken to enable movement of the activation lever.

17. The volatile composition dispenser according to claim 16, wherein the fixation element is broken by the application of a pressure onto the handle portion of the activation lever through which pressure the activation lever could pivot from the first position to the second position.

18. The volatile composition dispenser according to claim 1, wherein the cartridge further comprises a breathable membrane for enclosing the reservoir and evaporating the at least one liquid volatile composition into the atmosphere.

19. The volatile composition dispenser according to claim 18, further comprising a rupture mechanism sandwiched between the rupture substrate and the breathable membrane, wherein upon the activation lever pivoting from the first position to the second position, the hammer portion of the activation lever exerts a force on the rupture mechanism and moves the rupture mechanism to rupture the rupturable substrate in the cartridge and to release the at least one liquid volatile composition therefrom.

20. A method of delivering at least one liquid volatile composition into the atmosphere, the method comprising:
(i) providing a volatile composition dispenser according to claim 1; and
(ii) activating the volatile composition dispenser by applying pressure onto the handle portion of the activation lever.
